(19) United States
(12) Patent Application Publication
(10) Pub. No.: US 2009/0202637 A1
(43) Pub. Date: Aug. 13, 2009

(54) ORAL PHARMACEUTICAL COATED COMPOSITION FOR PULSATILE RELEASE

(75) Inventor: Petra Gisela Rigassi-Dietrich, Therwil (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: Novartis AG, Basel (CH)

(21) Appl. No.: 12/293,206

(22) PCT Filed: Mar. 28, 2007
(86) PCT No.: PCT/EP07/52974

§ 371 Date: Sep. 16, 2008

(30) Foreign Application Priority Data
Mar. 31, 2006 (GB) 0606562.7

(51) Int. Cl. A61K 9/36; A61K 47/38

(52) U.S. Cl. 424/480; 514/781

(57) ABSTRACT

A pharmaceutical composition comprising a pharmaceutically active agent, a core, a coating comprising an inner film comprising cellulose acetate and hydroxypropylmethylcellulose in a ratio of cellulose acetate:hydroxypropylmethylcellulose of 80% to 99.5%:0.5% to 20% and an outer film comprising ethylcellulose and hydroxypropylcellulose in a ratio of ethylcellulose:hydroxypropylcellulose of 50% to 80%:20% to 50%.

The invention relates to a controlled release oral pharmaceutical composition and more particularly to a drug delivery system for pulsatile release.

Controlled release formulations may be formulated to control the time until the release of the active agent, also called lag time or delay time, the rate of release of active agent, e.g. slow or fast and the duration of release of active agent, e.g. long or short. Such aspects may be observed in standard in vitro dissolution tests, e.g. in water or if desired in body fluids, e.g. artificial gastric juices.

There are publications on time-controlled release formulations allowing a release with defined lag times, but there still exists a need for improved formulations which are reliable and commercially acceptable.

Surprisingly in the context of the present invention a pharmaceutical composition has now been found which is capable of releasing at a specific time, i.e., with time delay or lag time, a pharmaceutical active agent or an active agent mixture, e.g. substantially independent of the concentration and type of ions present in the gastrointestinal environment, e.g. hydrogen ions and hydroxyl ions, e.g., independently of pH, independently of phosphate ions, and also independently of enzymes, present into the surrounding body fluids.

The present invention provides in one aspect a pharmaceutical composition, preferably a tablet, comprising a pharmaceutically active agent, a core and a coating comprising an inner film and an outer film. The inner film may comprise cellulose acetate and hydroxypropylmethylcellulose in a ratio of cellulose acetate:hydroxypropylmethylcellulose of about 80% to about 99.5%:about 0.5% to about 20% and the outer film may comprise ethylcellulose and hydroxypropylcellulose in a ratio of ethylcellulose:hydroxypropylcellulose of about 50% to about 80%:about 20% to about 50%. Preferably, the inner film may comprise e.g. cellulose acetate and hydroxypropylmethylcellulose in a ratio of ethylcellulose hydroxypropylcellulose of about 98%:about 2%, e.g., about 95%:about 5%. Preferably, the outer film may comprise ethylcellulose and hydroxypropylcellulose in a ratio of e.g., ethylcellulose:hydroxypropylcellulose of about 70% to about 30%, e.g. about 60% to about 40%. In the context of the present invention it has been found that a smaller variation of the lag time may be obtained with such an embodiment. The inner film may be directly in contact with the core and may be a semi-permeable membrane, only permeable for water or body fluid but not for other substances. The brittle inner film may be cracked, leading to an opening of the system and starting of drug release from the core. The inner film may be applied as e.g. a 7% suspension in a solvent system acetone/ethanol/water. The outer film may be semi-permeable as well, e.g., allowing the passage of water, or permeable. The outer film may control the permeation of water into the core, resulting in a targeted opening time of the system. The outer film may be applied as a 5% suspension in a solvent system of e.g. acetone/ethanol/ (60/40).

The core of the pharmaceutical composition may comprise the active agent, optionally a disintegrant and/or optionally an osmotic agent. Preferably, the core of the pharmaceutical composition may comprises the active agent, a disintegrant and optionally an osmotic agent. In one aspect of the invention, the active agent may act as the osmotic agent and/or as disintegrant. The osmotic agent may adsorb water or body fluid through the permeable outer film and the inner film. The disintegrant swells in the presence of water or body fluid and creates mechanical pressure causing the coating to rupture or break and the system to open, e.g. like a lid of a box.

By “in vitro opening time” is meant the time period from first contact with water until the rupture of the film coat and release of the active agent from the tablet.

By “lag time” is meant the duration of the time between administration of the composition and the release of an effective dose of active agent from the core.

By “semi-permeable membrane” is meant a membrane suitable for the passage of the water, e.g. from body fluid, into an active agent containing core which is coated with said membrane and hinders egress of a dissolved active agent out of the core.

The advantages of the present invention are that defined lag times may be achieved by the amount of outer film applied to the core. The outer ethylcelluose and hydroxypropylmethylcellulose coat controls the permeation of water into the core, resulting in the targeted opening time of the system. A further advantage is that the combination of both film coats, the inner and the outer, leads to a significant reduction of total film weight and to a reproducible cracking of the film compared to only one film coat.

The composition according to the present invention may be used for administering a wide variety of active agents, such as solid, pharmaceutical active ingredients, which may be inorganic or, in particular, organic active substances, and are to be used in accordance with their indication as analgesics, antipyretics, antiasthmatics, antirheumatics, sedatives, hypnotic agents, anti-epileptics, depressants and stimulants, anaesthetics, neuroleptic analgesics, antihistamines, antihypertensive agents, anticoagulants, antithrombotic agents, psychopharmacological agents, psycholeptics, chemotherapeutic agents, e.g. antibiotics, sulphonamides, antituberculosis agents (tuberculostatic agents) or also chemotherapeutic agents against tropical infections, diuretics, spasmolytics, cardiovascular agents, e.g. sympathomimetics, antihypertensive agents, cardiac stimulants, e.g. cardiac glycosides and digitaloids, parenteral sugar therapeutics, analeptics acting on the central nervous system, geriatric agents, tonolytics (of striated muscles), anti-Parkinson agents, cytostatic agents, immunosuppressants, tonics and vitamins, according to B. Helwig (Moderne Arzneimittel), 1980, or antibiotics, penicillin, tetracycline, chlorotetracycline, bacitracin, nystatin, streptomycin, neomycin, polymicin, gramicidin, oxytetracyclin, chloramphenicol, erythromycin, rifampicin, cefazolin, cefoxitin, cefsulodin, cefotiam and mefoxin may be used, and as chemotherapeutic agents sulfamethazine, sulfamerazine, sulfamethizole and sulfisoxazole may be used, as solid active ingredients for the presentation according to the invention. In addition, e.g. as sedatives and hypnotic agents chloral hydrate, pentabarbital, phenobarnital, secobarbital, codeine and carbromal may be used, and as cardiac glycosides and digitaloids digitoxin and digoxin may be used, and as sympathomimetics epinephrine may be used as the solid active substance in water-soluble form or water-insoluble form.

In particular, antipyretics, analgesics and antirheumatics may be used as the solid active ingredient in the presentation according to the invention in suitable water-soluble form or water-insoluble form, for example propyphenazone, aminophenazone, aspirin (ASA), antipyrine, methyl nifenazine, melaminsulfone, sulfenazone, phenacetin, pentazocine, lactophenin, paracetamol, quinine, flufenamic acid, mefenamic acid, tolfenamic acid, meclofenamic acid, niflumic acid, clonixin or clonixidin, flunixin, ibuprofen, suprofen, ketoprofen, fenoprofen, pirprofen, diclofenac, ibufenac, procticic acid, naproxen, cicloprofen, tolmetin, clopirac, tiaprofenic acid, oxaprozin, fenclozic acid, fentiazac, clidanac, fenclonac, fenoprofen, flurbiprofen, carprofen, sulindac, cinmetacin, fenbuten, etodolac, butifufen.

Most advantageously, psychopharmacological agents may be used as the solid active ingredient according to the invention, e.g. neuroleptics, antidepressants, thymoleptics, thymerethical drugs and tranquilisers in water-soluble form or water-insoluble form, such as thioridazine, imipramine, desimipramine, clomipramine, ketimipramine, opipramol, amitriptyline, nortriptyline, reserpine, aromazine, chlorpromazine, fluopromazine, methopromazine, trimeprazine, diethazine, promethazine, aminopromazine, mepazine, pipamazine, maprotiline and rivastigmine.

In addition, antihypertensive agents, such as oxprenolol and metoprolol may be used as the solid active ingredient in the compositions of the invention.

The active agent of the present invention may have a solubility in water of from about 0.1 mg/ml, e.g., 0.5 mg/ml, e.g., 1 mg/ml to about 750 mg/ml, preferably from about 9 mg/ml to about 500 mg/ml.

The amount of active agent according to the present invention may be from about 1 mg to about 150 mg, preferably from about 1 mg to about 100 mg, most preferably about 1.5 mg to about 81 mg compound per unit dosage from.

The core of the composition of the present invention may contain a binder. Suitable binders are polysaccharides, e.g. potato starch, wheat starch, corn starch, cellulose and derivatives thereof, for example microcrystalline cellulose, e.g., a product known under the registered trade marks Avicel PH (Fiedler, loc.cit., p. 275), hydroxypropyl cellulose, hydroxyethyl cellulose, hydroxypropylmethylcellulose, sucrose, dextrose and gelatin. The binder may be present in an amount from about 0% to about 50%, e.g. about 0.5% to about 20%, or about 0.7% to about 15%, e.g. about 0.7% to about 14.6% by weight of the composition.

The core of the present invention may further comprise a disintegrant. As disintegrant the composition may comprise starch, clay, cellulose, gum, a crosslinked or non-crosslinked polymer, e.g., polyvinyl polypyrrolidone, cross-linked polyvinyl polypyrrolidone or crospovidone, e.g., Polyplasdone XL from International Speciality Products (Wayne, N.J.), cross-linked sodium carboxymethylcellulose or croscarmellose sodium, e.g., AC-DI-SOL from FMC, and cross-linked calcium carboxymethylcellulose, soy polysaccharides, and guar gum. The disintegrant may be present in an amount from about 1% to about 75%, preferably from about 50% to about 60% by weight of the composition.

The core of the composition may further comprise an osmotic substance or agent, e.g., any salt, preferably sodium chloride. The active agent itself may have osmotic properties and may serve as the osmotic substance. The osmotic agent may be present in an amount from about 1 % to about 50%, preferably from about 20% to about 30% by weight of the composition.

The core of the composition of the present invention may further comprise a lubricant. Examples of lubricants include magnesium stearate, aluminium stearate, calcium stearate, magnesium carbonate, magnesium oxide, sodium benzoate, glycerol mono fatty acid, e.g. having a molecular weight of from 200 to 800 Daltons, e.g., glycerol monostearate (e.g. Danisco UK), glycerol dibehanate (e.g., CompritolATO888™, Gattefossë France), glycerol palmito-stearic ester (e.g. Precirol™, Gattefossë France), polyethylene glycol, hydrogenated cotton seed oil (Lubitrab, Edward Mendell Co Inc), castor seed oil (Cutina HR, Henkel). The lubricant may be present in an amount from about 0.1% to about 5%, e.g. from about 0.1% to about 3% or, e.g., from about 0.2% to about 1% by weight of the composition.

The core of the composition of the present invention may further comprise a glidant. Examples of glidants include colloidal silicon dioxide, e.g., as known under the registered trademark Aerosil (Fiedler loc.cit. p. 161) and talc. The glidant may be present in an amount from about 0.1% to about 10%, e.g. from about 2 to about 6% by weight of the composition.

Other excipients disclosed in the literature, as for instance in Fiedler's “Lexikon der Hilfstoffe”, 5^{th} Edition, ECV Aulendorf 2002 and “Handbook of Pharmaceutical Excipients” Wade and Weller 4^{th} Edition 2003, the contents of which are incorporated herein by reference, may be used in the pharmaceutical compositions according to the invention.

According to the present invention the core may be coated by film-coating, e.g., by an inner film which is semi-permeable to water and body fluid and an outer film which is permeable. The combination of the two coatings may provide for the pulsatile release of the active agent.

The inner film may comprise cellulose acetate and hydroxypropylmethylcellulose. The cellulose acetate may be, e.g., cellulose acetate E320 or 398-10 (Handbook of Pharmaceutical Excipients loc.cit. p. 117). The hydroxypropylmethylcellulose has preferably a viscosity of about 3 cps (2% (w/w) aqueous solution).

The outer film coating may comprise ethylcellulose and hydroxypropylmethylcellulose. The ethylcellulose may be, e.g., ethylcellulose known and registered as Aqualon N10 (available from Dow Chemicals Company). The hydroxypropylmethylcellulose has preferably a viscosity of about 3 cps (2% (w/w) aqueous solution).

The coat weight of the inner cellulose acetate film may be in a range of about 1 to about 20 mg/cm² and the coat of the outer film may be in a range of about 1 to 15 mg/cm².

In one embodiment the present invention provides a oval shaped tablet or an oval shaped mini-tablet with a tablet size of 5×1.9 millimeters to 22×8.7 millimeters, preferably 10×5.2 millimeters, 14×5.5 millimeters, and 17.5×7 millimeters. Minitablets may be filled in a sachet or a capsule.

The drug loading of a tablet core of the present invention may range from about 0.01% to about 10% for low-dose tablet cores and from about 10% to about 50% for moderate-dose tablet cores. The drug load may be up to about 96% if the drug substance works as a disintegrant.

The opening time of the tablet may be determined by standard dissolution tests using conductometric measurement. The tablets may be in a standard dissolution apparatus filled with 1 Liter demineralized water at 37 degrees Celsius including a rotary paddle stirring at a rate of 50 rpm. An electrode may continually measure the conductivity of the dissolution medium. The tablets of the present invention may contain sodium chloride or any other conductive substance in the core as the osmotic agent which may be after opening of the tablet released into the dissolution medium. The dissolved sodium chloride may increase the conductivity of the dissolution medium. This increase is measured and the time of the increase is registered.

The opening time of the tablet may vary between a minimal opening time of 1 hour and a maximal opening time of 14 hours. The opening time is dependent on the drug loading of the core and the coat weight of the inner and the outer film.

In a further aspect the present invention provides a process for the production of the compositions of the invention.

Process

The composition of the invention may be obtained by
(i) dissolving the active agent in purified water
(ii) mixing of, if present, the disintegrant, binder, osmotic agent and the glidant
(iii) mixing mixture (i) with mixture (ii)
(iv) drying the granules, e.g. in fluidized bed
(v) sieving the dried granules, e.g. through a 800 micrometer sieve
(vi) mixing the dried granules of step (v) with the lubricant and the glidant
(vii) forming the composition.

Preferably, the composition of the invention may be obtained by
(i) dissolving the active agent in purified water
(ii) mixing of, if present, the disintegrant, binder, osmotic agent and the glidant
(iii) mixing mixture (i) with mixture (ii)
(iv) drying the granules, e.g. in fluidized bed
(v) sieving the dried granules, e.g. through a 800 micrometer sieve
(vi) mixing the dried granules of step (v) with the lubricant and the glidant
(vii) forming tablets, e.g. by direct compression.

Alternatively the composition of the invention may be obtained by the following process
(i) dry blending of the active substance with, if present, the disintegrant, binder, osmotic agent and the glidant
(ii) addition of a granulation liquid
(iii) drying the granules, e.g. in fluidized bed
(iv) sieving the dried granules, e.g. through a 800 micrometer sieve
(v) mixing the dried granules of step (iv) with the lubricant and the glidant
(vi) forming the composition.

Preferably, alternatively the composition of the invention may be obtained by the following process
(i) dry blending of the active substance with, if present, the disintegrant, binder, osmotic agent and the glidant
(ii) addition of a granulation liquid
(iii) drying the granules, e.g. in fluidized bed
(iv) sieving the dried granules, e.g. through a 800 micrometer sieve
(v) mixing the dried granules of step (iv) with the lubricant and the glidant
(vi) forming tablets, e.g. by direct compression.

The final composition, in particular tablets, preferably compressed tablets that have been manufactured by these processes may be coated by an inner and an outer coating.

A preferred composition according the present invention may comprise as an active agent, e.g. rivastigmine, e.g., (weight %):

Core

| | |
|---|---|
| Rivastigmine hydrogen tartrate | 0.5 to 25% |
| Sodium Chloride | 10 to 35% |
| Avicel PH 102 | 0.5 to 25% |
| PVPP-XL | 20 to 70% |
| Aerosil 200 | 1 to 10% |
| Magnesium Stearate | 0.1 to 5% |

Inner Film Coating:

| | |
|---|---|
| Cellulose Aceate | 80 to 99.5% |
| HPMC | 0.5 to 20% |

Outer Film Coating:

| | |
|---|---|
| Ethylcellulose | 50 to 80% |
| HPMC | 20 to 50% |

The compositions of the invention with a pulsed drug release may be useful in order to achieve a once daily dosing of active agents. Compositions of the invention may be useful for the treatment of coronary infarctions, angina pectoris attacks, bronchial asthma, rheumatic pains or mild to moderately severe dementia of the Alzheimer type, also known as Alzheimer's Disease.

The following examples are illustrative, but do not serve to limit the scope of the invention described herein. The examples are meant only to suggest a method of practicing the present invention.

### EXAMPLE 1

A. Preparation of the Core:

The mass for 60,000 cores is prepared as follows. 144 g of rivastigmine hta is dissolved in about 9300 g of purified water. 5947 g of Polyplasdone-XL, 1576 g of Microcrystalline Cellulose, 2652 g of Sodium Chloride (previously milled), and 274 g of colloidal silicon dioxide (Aerosil 200) are transferred into a 75 L Collette Gral High Shear Mixer. In the Collette Gral the dry powders are mixed for three minutes with Impeller at slow speed and Chopper off. After that the rivastigmine solution is added slowly (2 L/min dosage rate) with the Impeller and Chopper both operating at a slow speed. The Collette Gral is operated for three minutes with the Impeller and Chopper at slow speeds. Then the granules are dried in the fluidized bed dryer with inlet air temperature of about 70° C., till a Loss on Drying of less then 3% is achieved. After that the dried granules are sieved through an 800 μm sieve and mixed with the magnesium stearate and colloidal silicon dioxide (Aerosil 200) (both previously sieved through a 0.5 mm sieve size) for 120 rotations in a free fall blender.

B. Tabletting

This mixture is then compressed into tablets of 178 mg using oblong tooling of size 10×5.2 mm using a suitable tablet press. Core hardness is 130 N.

C. Film Coating:

First the two solutions for the two films are prepared. 428 g of Cellulose Acetate 398-10, 428 g of Cellulose Acetate 320S and 45 g of 3 cps HPMC are dissolved in a solvent mixture of 70% Acetone, 20% Ethanol and 10% Purified Water to form a 7.5% solution by weight of solid components. 216 g of Ethyl Cellulose 10 cps and 144 g of 3 cps HPMC are dissolved in a solvent mixture of 60% Acetone and 40% Ethanol to form a 5% solution by weight of the solid components. Up to 20% extra solution may be prepared to account for the loss from spray drying during the coating process. The tablets prepared above are coated in a suitable Perforated Coating Pan by spraying first the Cellulose Acetate solution and then the Ethyl Cellulose solution, to target film weights. Other solvent systems such as methylene chloride/methanol may also be used.

C. Compositions

| | |
|---|---|
| Ingredients | Quantity/tablet (mg) |

| | | |
|---|---|---|
| Rivastigmine hta | 2.4 | 2.4 |
| Sodium Chloride | 44.2 | 44.2 |
| Avicel PH 102 | 26.27 | 26.27 |
| PVPP-XL | 99.11 | 99.11 |
| Aerosil 200 | 5.02 | 5.02 |
| Magnesium Stearate | 1.0 | 1.0 |
| Core Weight | 178.0 | 178.0 |
| Cellulose Aceate 398-10 | 7.125 | 7.125 |
| Cellulose Acetate E320 | 7.125 | 7.125 |
| HPMC 3 cps | 0.750 | 0.750 |
| Ethylcellulose 10 cps | 3.6 | 7.2 |
| HPMC 5 cps | 2.4 | 4.8 |
| Total Weight | 199 | 205 |

### EXAMPLE 2

Composition of Tablet Cores

| | Ingredient | | | |
|---|---|---|---|---|
| Component | Rivastigmine | Rivastigmine | Diclofenac | Diclofenac |

Inner phase (granulated)

| | | | | |
|---|---|---|---|---|
| Rivastigmine | 1.3% | 15.3% | | |
| (tartrate) | | | | |
| Diclofenac | | | 1.4% | 15.3% |
| sodium | | | | |
| Avicel PH 101 | 14.8% | 0.8% | 14.8% | 0.8% |
| Polyvinylpoly- | 55.7% | 55.7% | 55.7% | 55.7% |
| pyrrolidone XL | | | | |
| Sodium Chloride | 24.8% | 24.8% | 24.8% | 24.8% |
| Aerosil 200 | 2.6% | 2.6% | 2.6% | 2.6% |

Outer phase

| | | | | |
|---|---|---|---|---|
| Aerosil 200 | 0.3% | 0.3% | 0.3% | 0.3% |
| Magnesium | 0.6% | 0.6% | 0.6% | 0.6% |
| stearate | | | | |

1. A pharmaceutical composition comprising a pharmaceutically active agent, a core, a coating comprising an inner film comprising cellulose acetate and hydroxypropylmethylcellulose in a ratio of cellulose acetate:hydroxypropylmethylcellulose of 80% to 99.5%:0.5% to 20% and an outer film comprising ethylcellulose and hydroxypropylcellulose in a ratio of ethylcellulose:hydroxypropylcellulose of 50% to 80%:20% to 50%.

2. The pharmaceutical composition according to claim 1 wherein the core comprises the pharmaceutically active agent.

3. The pharmaceutical composition according to claim 2 wherein the core further comprises a disintegrant.

4. The pharmaceutical composition according to claim 2 wherein the core further comprises an osmotic agent.

5. The pharmaceutical composition according to claim 1 wherein the amount of active agent is from 1 to 150 mg per dosage form based on the total weight of the dosage form.

6. The pharmaceutical composition according to claim 1 wherein the solubility of the active agent in water is from 0.5 to 750 mg/ml.

7. The pharmaceutical composition according to claim 3 wherein the disintegrant is polyvinyl polypyrrolidone.

8. The pharmaceutical composition according to claim 4 wherein the osmotic agent is a salt.

9. The pharmaceutical composition according to claim 8 wherein the osmotic agent is sodium chloride.

10. The pharmaceutical composition according to claim 1 wherein the core further comprises a binder.

11. The pharmaceutical composition according to claim 10 wherein the binder is microcrystalline cellulose, hydroxypropyl cellulose, hydroxyethyl cellulose, or hydroxypropylmethylcellulose.

12. The pharmaceutical composition according claim 1 wherein the core comprises a glidant.

13. The pharmaceutical composition according to claim 12 wherein the glidant is colloidal silicon dioxide.

14. The pharmaceutical composition according to claim 1 wherein the core comprises a lubricant.

15. The pharmaceutical composition according to claim 14 wherein the lubricant is magnesium stearate.

16. A process for the production of a composition as claimed in claim 1 which comprises
(i) dissolving the active agent in purified water
(ii) mixing of, if present, the disintegrant, binder, osmotic agent and the glidant
(iii) mixing dissolution (i) with mixture (ii)
(iv) drying the granules, e.g. in fluidized bed
(v) sieving the dried granules, e.g. through a 800 micrometer sieve
(vi) mixing the dried granules of step (v) with the lubricant and the glidant
(vii) forming the composition.

17. A process for the production of a composition as claimed in claim 1 which comprises
(i) dry blending of the active agent with, if present, the disintegrant, binder, osmotic agent and the glidant
(ii) addition of a granulation liquid
(iii) drying the granules, e.g. in a fluidized bed
(iv) sieving the dried granules, e.g. through a 800 micrometer sieve
(v) mixing the dried granules of step (iv) with the lubricant and the glidant
(vi) forming the composition.
